(19) United States
(12) Patent Application Publication
(10) Pub. No.: US 2022/0372308 A1
(43) Pub. Date: Nov. 24, 2022

(54) RETICULATED CARBON COMPOSITES

(71) Applicant: Arkema Inc., King of Prussia, PA (US)

(72) Inventors: Ramin AMIN-SANAYEI, King of Prussia, PA (US); Jeremie BREZUN, Marseille (FR); Mark AUBART, King of Prussia, PA (US)

(21) Appl. No.: 17/616,802

(22) PCT Filed: Jun. 18, 2020
(86) PCT No.: PCT/US2020/038405

§ 371 Date: Dec. 6, 2021

Related U.S. Application Data
(60) Provisional application No. 62/863,495, filed on Jun. 19, 2019.

(51) Int. Cl. C09D 7/61; C09D 7/40; C09D 133/12; C09D 127/16; C08J 5/18; C08J 9/00; C08J 9/28

(52) U.S. Cl. CPC C09D 7/61; C09D 7/68; C09D 133/12; C09D 127/16; C08J 5/18; C08J 9/008; C08J 9/28; B82Y 30/00

(57) ABSTRACT

This invention discloses a reticulated film composite and a method of fabricating a reticulated film composite suitable as a 3 dimensional porous and conductive matrix which contains up to 80% porosity and exhibits high recovery after compression. The reticulated film composite is produced by casting and drying of a slurry which exhibits a high yield stress (i.e. greater than 50 dyne/cm2) and comprised of a high MW resin dissolved in a solvent (i.e. having solution viscosity of higher than 100 cp at 5% in NMP at room temperature) and dispersed nanoparticles of carbon of high specific surface areas (i.e. greater than 1 m2/g preferably greater than 10 m2/g), examples include but not limited to conductive carbon, carbon nanotubes, graphene, activated carbon or mixture thereof. This reticulated film composite exhibits high electrical conductivity (i.e. volume resistivity of less than 10,000 Ω·cm) and superior dimensional stability even at elevated temperatures (i.e. at 140° C.). It will exhibit a recovery of height or porosity after being compressed to over 50% of its height. The composite of this invention is suitable as an electrically conductive composite, as a gas diffusion layer in a fuel cell, or as a high efficiency electrode in super capacitors

### FIELD OF THE INVENTION

This invention discloses a method of fabricating a reticulated (porous, open cell matrix structure) film composite and said composite suitable as an electrically conductive composite, as a gas diffusion layer in a fuel cell, or as a high efficiency electrode in super capacitors.

### BACKGROUND

Reticulated films composites are very porous, low density solid films. Reticulated foams are refer to a very open structure like a net. Similarly, reticulated film composites are also made of an extremely open cell structures where exhibit novel physical properties compared with their bulk counterparts such as large specific area and high energy absorption at impact. Its outstanding strength to weight ratios make them ideal for catalysis media, catalyst support, energy storage, damping, building structural components, and protective coatings. However, when sufficient amount of carbon (>20%) is incorporated into the solid film, in order to impart electrical conductivity, the film often exhibits poor mechanical and thermal stabilities. It would have much lower elongation at break which translate into being brittle and breakable and not being transformable to foam or reticulated film.

Thin porous films are often made of melt processable plastics, which are either solution cast or extruded to form films and then stretched to generate 30-60% porosity within the film. Today's common porous thin films (less than 100 micron thick) are generally based on polypropylene (melting point about 160-165° C.), polyethylene (melting point about 110-135° C.) or blends thereof. For example U.S. Pat. Nos. 4,620,956 and 5,691,047 describe melt extrusion and stretch process to make polyolefin porous film or separators, and U.S. Pat. Nos. 8,064,194 and 8,012,799 disclose solution cast process for producing polyolefin porous film or separators. Also known are porous separators made of poly-vinylidene fluoride, PVDF, (melting temperature about 165-170° C.) disclosed in the US patent applications of 2009/0208832 and 2010/0183907. A serious drawback of such porous thin films is exhibits a poor dimensional stabilities at elevated temperature or a lack of thermal robustness which may lead to shrinkage. Moreover, to the best of the author's knowledge there is no porous conductive thin film (less than 100 micron thick) with porosity greater than 20% and volume resistivity of less than 10,000 Ω·cm.

PVDF has been found to be useful as a binder or coating for separators in non-aqueous electrolytic devices because of its excellent electro-chemical resistance and superb adhesion among fluoropolymers. The separator forms a barrier between the anode and the cathode in the battery to prevent electronic shorts while allowing high ionic transportation. Poly vinylidene fluoride, PVDF, and its copolymers have been used in many applications, such as durable coating, wire jacketing, binder in lithium ion battery, chemicals piping, open and closed cell foams. However, it exhibits high insulting properties and requires over 30% carbon to become conductive. At such high carbon loadings, it is next to impossible to make low density foams or films out of PVDF-carbon composites.

### DESCRIPTION OF THE FIGURES

FIG. 1 is a high-resolution picture of a composite of this invention made of PVDF/carbon at ratio of 40/60 cast from a slurry having 4% solids where the carbon is Denka Black Li435 (from Denka) and PVDF is Kynar® HSV-1810 (from Arkema). The composite was dried in oven at 120 C for 30 min.

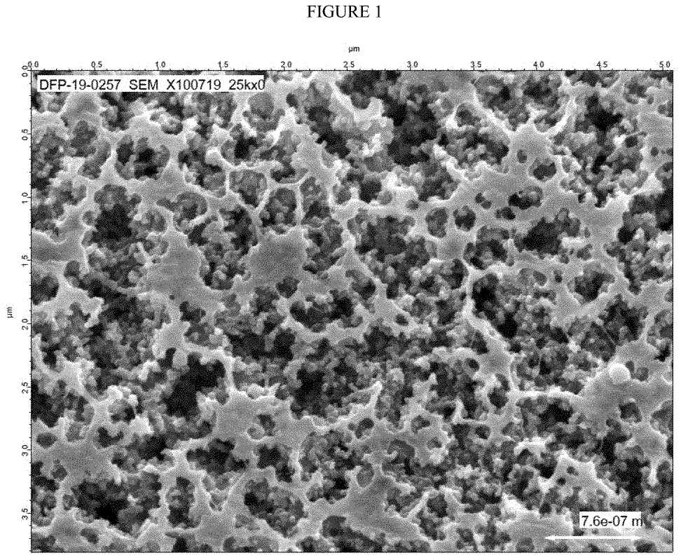

### DESCRIPTION OF THE INVENTION

“Copolymer” is used to mean a polymer having two or more different monomer units. “Polymer” is used to include homopolymer and copolymers. Resin and polymer are used interchangeably. The polymers may be homogeneous, heterogeneous, and may have a gradient distribution of co-monomer units. All references cited are incorporated herein by reference. As used herein, unless otherwise described, percent shall mean weight percent. Crystallinity and melting temperature are measure by DSC as described in ASTM D3418 at heating rate of 10 C/min. Melt viscosity is measured in accordance with ASTM D3835 at 232° C. expressed in kPoise @100 Sec−1 . Dilute solution viscosity and reduced viscosity of polymers is measured as described in ASTM D2857 at room temperature.

By reticulated film or coating we mean a film or coating with a porous open cell matrix structure. “Open cell” means the pores are not enclosed. Fluids can moves between pores. The void fraction or porosity can be measured by compressing the open cell matrix, or by density measurement, or by filling the void with a liquid and measuring the change in density. Preferably the voids are measured by density.

Nano sized filler or nano size particles means that the filler or particle size is less than 1 micron, preferably less than 500 nm preferably less than 200 nanometers. The nano size particles can be less than 100 nm. Particles size is volume average particles size as measured by light scattering. (such as a Nicom or Microtech instrument).

By high specific surface area particles means that the surface area of the particles is greater than 1 m²/g, preferably greater than 5 m²/g, more preferably greater than 10 m²/g . Preferably between 1 m²/g and 10000 m²/g, preferably between 1 m²/g and 5000 m²/g, between 1 m²/g and 1000 m²/g, more preferably between 1 m²/g and 700 m²/g, and even more preferably between 10 m²/g and 500 m²/g. The surface area of the particles can be between 5 m²/g and 700 m²/g. Some high specific surface area particles have 3 dimensional branching structures, this can be referred to as a fractal shape which can result in particles with large aspect ratios. Fractal shape are aggregates that have 3 dimensional branching. For example primary particle of Conductive carbon structure can agglomerate into a 3 dimensional branching structure, which is made of many primary particles that are tightly bound together

By high molecular weight means having solution viscosity of at least 100 cp measured at 5% in NMP at room temperature (25° C.), preferably between 100 cp and 10,000 cp, more preferably between 100 cp and 5000 cp or having reduced viscosity, Rv of at least 0.2 dl/g up to 2 dl/g measured using ASTM D2857.

Yield stress is the minimum shear stress required to initiate flow in a fluid. A high yield stress is at least 50 dyne/cm² preferably greater than 100 dyne/cm2, greater than 125 dyne/cm2. The yield stress can be up to 5000 dyne/cm2, preferably up to 3000 dyne/cm2. In addition, the slurry must be castable meaning the solution viscosity of the slurry is less than 20,000 cP at room temperature, preferably less than 10,000 cp.

Recovery of volume or porosity after being compressed and then heated is calculated by dividing the thickness of the coating or film after being compressed and then heated for 10 minutes at 150 C by the thickness prior to compression.

The invention provides for a reticulated film composite with nano sized pores and a method of making the reticulated film composite with nano sized pores. Nanosized pores have an average pore size of less than 500 nm, preferably from 2 nm to 500 nm. The invention also provides for a coating made from the reticulated film composite with nano sized pores that after compression has a recovery of porosity to at least 30% of the porosity before compression. The recovery of volume or porosity after being compressed and then heated can be at least 30%, preferably 50%, preferably 55%, preferably 60%, preferably 70% or the original thickness.

The reticulated film composites can be produced with different type of resins and wide variety of carbon based nano-size particles.

The reticulated film composite is made by combining high specific surface area particles and high molecular resins in solvent at room temperature (25° C.) resulting in a slurry that exhibits a high yield stress (greater than 50 dyne/cm²) even at low solid content (i.e. total solids less than 30 weight %, preferably less than 20 wt %, more preferably less than 12% or even less than 10%). Casting the slurry and drying at elevated temperatures thereby forming a reticulated film composite with nano sized pores. The film after compression when heated (between 30 to 180° C., preferably above 80° C., more preferably above 110 C) has a porosity recovery to at least 30% of the original porosity prior to compression; preferably at least 60%, preferably 50%, preferably 55%, even more preferably at least 70% of the original porosity prior to compression.

Surprisingly, it was found that a slurry of a high specific surface area particles (i.e. nanosized carbon based material such as conductive carbon, carbon nanotubes, graphene) and a high molecular resins, (for example, high MW-PVDF (having solution viscosity of greater than 100 cp at 5% in NMP at room temperature), or high MW-PMMA(having reduced viscosity, Rv of greater than 0.5 dl/g), which are made in NMP can exhibit high yield stress (greater than 50 dyne/cm²) even at low solid content (i.e. total solids less than 30 weight %, preferably less than 20 wt %, more preferably less than 12% or even less than 10%). It is easy to cast due to low dispersion viscosity (i.e. less than 10,000 cp at room. When this high yield stress slurry was cast and dried at elevated temperatures, (i.e. 50 to 180 C, preferably 80 to 180° C., preferably above 120° C.), a reticulated film composite with nano sized pores was formed. The film exhibited a recovery of porosity after compression when heated. Interestingly, these reticulated film composites can be compressed to half of their thicknesses using a room temperature calendaring rolls; which simply indicates that the composites contain at least about 50% porosity. More unexpectedly, the compressed composites can be expanded back to more than 50% of their original height when a simple polymer relaxation take place, such as for example placing them in an oven at 120° C. or exposing them to latent solvents. The bounce back indicates that these composites have an open pore structure which are mechanically very persistent. The ratio of carbon to polymer can vary vastly and a higher carbon content gave a higher porosity (lower density) composites.

The carbon filler type can be for example conductive carbon, carbon nanotubes, graphene or combination thereof to impart high electronic conductivity.

In one embodiment of the invention, high molecular weight PVDF (with solution viscosity of greater than 100 cp measured at 5% in NMP at room temperature) which is semi-crystalline works in the invention.

High molecular weight resin like PMMA (with reduced viscosity, Rv, of greater than 0.5 dl/g), and also high MW PAA (with solution viscosity of from 100 and up to 10000 cp, preferably up to 5000 measured in water at pH 7 at room temperature) can be used to obtain a high yield stress slurry (greater than 50 dyne/cm²), and ultimately produce the reticulated film composites of similar properties to reticulated film made with PVDF.

The filler type useful in the invention are carbon based materials for example, include, but not limited to conductive carbon, carbon nanotubes, activated carbon, graphene or combination thereof

Small amounts of other filler (0 to 15 wt %, preferably less than 10 weight %) can be present in the composition the other filler include for examples alumina, silica, BaTiO₃, CaO, ZnO, bohemite, TiO₂, SiC, ZrO₂, boron silicate, BaSO₄, nano-clays, Pb(Zr,Ti)O₃, Pb₁₋ₓ, LaₓZr_{y}O₃ (0<x<1, 0<y<1), PBMg₃Nb_{2/3})₃, PbTiO₃, hafnia (HfO(HfO₂), SrTiO₃, SnO₂, CeO₂, MgO, NiO, Y₂O₃, Al₂O₃, SiO₂, ceramics, or mixtures thereof. Also, other useful organic fillers are chopped fibers, include, but not limited to aramid fillers and fibers, polyetherether ketone fibers, polyetherketone ketone fibers, PTFE fibers, and nanofibers, carbon nano-tubes, and mixture thereof.

The resin should have a high solution viscosity, i.e. higher than 100 cp measured at 5% in NMP at room temperature. Preferably, the solution viscosity is between 100 and 10,000 cp, more preferably between 100 and 5000 cp measured at 5% solids in NMP at room temperature. For water soluble polymers the solution viscosity is from 100 cp to 10000 cp, preferably between 100 cp and 5000 cp measured in water at 2% and pH of 7 at room temperature (25° C.). For this application, the pH can vary from 2 to 12 depending on polymer type and application.

Polymers (resins) useful in the invention include but not limited to homopolymers and copolymers of polyvinylidene fluoride (PVDF), poly ethylene-tetrafluoride ethylene (PETFE), polyvinyl fluoride (PVF), poly (alkyl)acrylates, poly (alkyl)methacrylates, poly styrene, poly vinyl alcohol (PVOH), polyesters, polyamides, poly acrylonitrile, poly acrylamide, carboxymethyl cellulose CMC, polyacrylic acids (PAA), polymethacrylic acids (PMAA). Other useful polymers include polyether ketone ketone, polyether ether ketone, and polyesters.

### Polyvinylidene Fluoride

In a preferred embodiment, the polymer is a polyvinylidene fluoride homopolymer or copolymer. The term “vinylidene fluoride polymer” (PVDF) used herein includes both normally high molecular weight homopolymers, copolymers, and terpolymers within its meaning. Copolymers of PVDF are particularly preferred, as they are softer—having a lower Tm, melting point and a reduced crystalline structure. Such copolymers include vinylidene fluoride copolymerized with at least one comonomer. Most preferred copolymers and terpolymers of the invention are those in which vinylidene fluoride units comprise at least 50 mole percent, at least 70 mole percent preferably at least 75 mole %, more preferably at least 80 mole %, and even more preferably at least 85 mole % of the total weight of all the monomer units in the polymer.

Copolymers, terpolymers and higher polymers of vinylidene fluoride may be made by reacting vinylidene fluoride with one or more monomers from the group consisting of vinyl fluoride, trifluoroethene, tetrafluoroethene, one or more of partly or fully fluorinated alpha-olefins such as 3,3,3-trifluoro-1-propene, 1,2,3,3,3-pentafluoropropene, 3,3,3,4,4-pentafluoro-1-butene, and hexafluoropropene, the partly fluorinated olefin hexafluoroisobutylene, perfluorinated vinyl ethers, such as perfluoromethyl vinyl ether, perfluoroethyl vinyl ether, perfluoro-n-propyl vinyl ether, and perfluoro-2-propoxypropyl vinyl ether, fluorinated dioxoles, such as perfluoro(1,3-dioxole) and perfluoro(2,2-dimethyl-1,3-dioxole), allylic, partly fluorinated allylic, or fluorinated allylic monomers, such as 2-hydroxyethyl allyl ether or 3-allyloxypropanediol, and ethene or propene. In some preferred embodiments the comonomer is selected from the group consisting of tetrafluoroethylene, trifluoroethylene, chlorotrifluoroethylene, hexafluoropropene, vinyl fluoride, pentafluoropropene, tetrafluoropropene, perfluoromethyl vinyl ether, perfluoropropyl vinyl ether.

Particularly preferred are copolymers composed of from at least about 75 and up to 90 mole percent vinylidene fluoride, and correspondingly from 10 to 25 mole percent hexafluoropropene. Terpolymers of vinylidene fluoride, hexafluoropropene and tetrafluoroethylene are also representatives of the class of vinylidene fluoride copolymers, embodied herein.

In one embodiment, up to 50%, preferably up to 20%, and more preferably up to 15%, by weight of hexafluoropropene (HFP) units and 50%, preferably 80%, preferably and more preferably 85%, by weight or more of VDF units are present in the vinylidene fluoride polymer. It is desired that the HFP units be distributed as homogeneously as possible to provide PVDF-HFP copolymer with excellent dimensional stability in an end-use environment—such as in a battery.

The copolymer of PVDF for use in the separator coating composition preferably has a high molecular weight as measured by melt viscosity. By high molecular weight is meant PVDF having a melt viscosity of greater than 10 kilopoise, preferably greater than 20 kilopoise, according to ASTM method D-3835 measured at 232° C. and 100 sec⁻¹.

Fluoropolymers such as polyvinylidene-based polymers are made by any process known in the art. Processes such as emulsion and suspension polymerization are preferred and are described in U.S. Pat. No. 6,187,885, and EP0120524.

### Synthetic Polyamides

A polyamide is a polymer (substance composed of long, multiple-unit molecules) in which the repeating units in the molecular chain are linked together by amide groups. Amide groups have the general chemical formula CO—NH. They may be produced by the interaction of an amine (NH₂) group and a carboxyl (CO₂H) group, or they may be formed by the polymerization of amino acids or amino-acid derivatives (whose molecules contain both amino and carboxyl groups).

The synthesis of polyamides is well described in the art, examples are WO15/071604, WO14179034, EP0550308, EP0550315, U.S. Pat. No. 9,637,595.

Polyamides can be condensation or ring opening products:
of one or more amino acids, such as aminocaproic, 7-aminoheptanoic, 11-aminoundecanoic and 12-aminododecanoic acid, or of one or more lactams such as caprolactam, oenantholactam and lauryllactam; with
of one or more salts or mixtures of diamines such as hexamethylenediamine, dodecamethylenediamine, meta-xylylenediamine, bis(p-aminocyclohexyl)methane and trimethylhexamethylenediamine with diacids such as isophthalic, terephthalic, adipic, azelaic, suberic, sebacic and dodecanedicarboxylic acid.

Examples of polyamides can include PA 6, PA 7, PA 8, PA9, PA 10, PA11, and PA 12 and copolyamides like PA 6,6.

The copolyamides can be from the condensation of at least two alpha, omega-amino carboxylic acids or of two lactams or of one lactam and one alpha,omega-amino carboxylic acid. The copolyamides can be from the condensation of at least one alpha,omega-amino carboxylic acid (or one lactam), at least one diamine and at least one dicarboxylic acid. Examples of lactams include those having 3 to 12 carbon atoms on the main ring, which lactams may be substituted. For example, of β,β-dimethylpropiolactam, α,α-dimethylpropiolactam, amylolactam, caprolactam, capryllactam and lauryllactam.

Examples of alpha,omega-amino carboxylic acids include aminoundecanoic acid and aminododecanoic acid. Examples of dicarboxylic acids include adipic acid, sebacic acid, isophthalic acid, butanedioic acid, 1,4-cyclohexanedicarboxylic acid, terephthalic acid, the sodium or lithium salt of sulphoisophthalic acid, dimerized fatty acids (these dimerized fatty acids having a dimer content of at least 98% and preferably being hydrogenated) and dodecanedioic acid, HOOC—(CH2)10-COOH.

The diamine can be an aliphatic diamine having 6 to 12 carbon atoms; it may be of aryl and/or saturated cyclic type. Examples include hexamethylenediamine, piperazine, tetra-methylenediamine, octamethylenediamine, decamethylenediamine, dodecamethylenediamine, 1,5-diaminohexane, 2,2,4-trimethyl-1,6-diaminohexane, diamine polyols, isophoronediamine (IPD), methylpentamethylenediamine (MPDM), bis(aminocyclohexyl)methane (BACM) and bis(3-methyl-4-aminocyclohexyl)methane (BMACM).

Examples of copolyamides include copolymers of caprolactam and lauryllactam (PA 6/12), copolymers of caprolactam, adipic acid and hexamethylenediamine (PA 6/6-6), copolymers of caprolactam, lauryllactam, adipic acid and hexamethylenediamine (PA 6/12/6-6), copolymers of caprolactam, lauryllactam, 11-aminoundecanoic acid, azelaic acid and hexamethylenediamine (PA 6/6-9/11/12), copolymers of caprolactam, lauryllactam, 11-amino-undecanoic acid, adipic acid and hexamethylenediamine (PA 6/6-6/11/12), and copolymers of lauryllactam, azelaic acid and hexamethylenediamine (PA 6-9/12).

Polyamides also include polyamide block copolymers, such as polyether-b-polyamide and polyester-b-polyamide.

Another polyamide is Arkema's ORGASOL® ultra-fine polyamide 6, 12, and 6/12 powders, which are microporous, and have open cells due to their manufacturing process. These powders have a very narrow particle size range that can be between 5 and 60 microns, depending on the grade. Lower average particle sizes of 5 to 20 are preferred.

### Acrylic

Acrylic polymers as used herein is meant to include polymers, copolymers and terpolymers formed from methacrylate and acrylate monomers, and mixtures thereof. The methacrylate monomer and acrylate monomers may make up from 51 to 100 percent of the monomer mixture, and there may be 0 to 49 percent of other ethylenically unsaturated monomers, included but not limited to, styrene, alpha methyl styrene, acrylonitrile. Suitable acrylate and methacrylate monomers and comonomers include, but are not limited to, methyl acrylate, ethyl acrylate and ethyl methacrylate, butyl acrylate and butyl methacrylate, iso-octyl methacrylate and acrylate, lauryl acrylate and lauryl methacrylate, stearyl acrylate and stearyl methacrylate, isobornyl acrylate and methacrylate, methoxy ethyl acrylate and methacrylate, 2-ethoxy ethyl acrylate and methacrylate, dimethylamino ethyl acrylate and methacrylate monomers. (Meth) acrylic acids such as methacrylic acid and acrylic acid can be comonomers. Acrylic polymers include multilayer acrylic polymers such as core-shell structures typically made by emulsion polymerization.

### Styrene

Styrenic polymers as used herein is meant to include polymers, copolymers and terpolymers formed from styrene and alpha methyl styrene monomers, and mixtures thereof. The styrene and alpha methyl styrene monomers may make up from 50 to 100 percent of the monomer mixture, and there may be 0 to 50 percent of other ethylenically unsaturated monomers, including but not limited to acrylates, methacrylates, acrylonitrile. Styrene polymers include, but are not limited to, polystyrene, acrylonitrile-styrene-acrylate (ASA) copolymers, styrene acrylonitrile (SAN) copolymers, styrene-butadiene copolymers such as styrene butadiene rubber (SBR), methyl methacrylate-butadiene-styrene (MBS), and styrene-(meth)acrylate copolymers such as styrene-methyl methacrylate copolymers (S/MMA).

Polyolefin as used herein is meant to include polyethyene, polypropylene, and copolymers of ethylene and propylene. The ethylene and propylene monomers may make up from 51 to 100 percent of the monomer mixture, and there may be 0 to 49 percent of other ethylenically unsaturated monomers, including but not limited to acrylates, methacrylates, acrylonitrile, anhydrides. Examples of polyolefin include ethylene ethylacetate copolymers (EVA), ethylene (meth)acrylate copolymers, ethylene anhydride copolymers and grafted polymers, propylene (meth)acrylate copolymers, propylene anhydride copolymers and grafted polymers.

The solvents useful in the invention to make the slurry include, but are not limited to water, N-methyl-2-pyrrolidone (NMP), toluene, tetrahydrofuran (THF), acetone and hydrocarbons. In preferred embodiments, the solvent is NMP, water, or acetone. The solvent must be able to dissolve the polymer used providing a visibly clear solution. For example, PVDF is soluble in NMP. PVDF is not soluble in water and therefore water would not be used for PVDF. Poly vinyl alcohol (PVOH), poly acrylamide, carboxymethyl cellulose CMC, Polyacrylic acids (PAA), and their copolymers are generally soluble in water.

### Other Additives:

The coating composition of the invention may further contain effective amounts of other additives, including but not limited to fillers, leveling agents, anti-foaming agents, pH buffers, and other adjutants typically used in formulation while meeting desired requirements.

In a slurry coating composition of the invention, could further optionally have wetting agents, thickeners or rheology modifiers.

Wetting agents could be present in the coating composition slurry at 0 to 5 parts (all parts by weight), or 0.1 to 5 parts preferably from 0 to 3 parts, or 0.1 to 3 parts of one or more wetting agents per 100 parts of solvent. Surfactants can serve as wetting agents, but wetting agents may also include non-surfactants. In some embodiments, the wetting agent can be an organic solvent. The presence of optional wetting agents permits uniform dispersion of powdery material(s) into the slurry. Useful wetting agents include, but are not limited to, ionic and non-ionic surfactants such as the TRITON series (from Dow) and the PLURONIC series (from BASF), BYK-346 (from BYK Additives)and organic liquids that are compatible with the solvent, including but not limited to NMP, DMSO, and acetone.

Thickeners and/or rheology modifiers may be present in the coating composition at from 0 to 10 parts (all parts by weight), preferably from 0 to 5 parts of one or more thickeners or rheology modifiers per 100 parts of water. Addition of thickener or rheology modifier to the above dispersion prevents or slows down the settling of powdery materials while providing appropriate slurry viscosity for a casting process. In addition to organic rheology modifiers, inorganic rheology modifiers can also be used alone or in combination.

The total solid content and ratio of resin to nano particle filler should be so chosen that provides a high yield stress slurry, i.e. higher than 50 dyne/cm², preferably greater than 75 dyne/cm2 even more preferably greater than 100 dyne/cm2 or even greater than 200 dyne/cm2. The yield stress can be up to 5000 dyne/cm2, preferably up to 3000 dyne/cm2.

The solids content of the slurry can be from 2 weight percent to 30 weight percent solids, preferably from 2 to 20 weight %, even more preferably from 2 to 12%, or 2 to 10 weight % (based on weight of polymer plus weight of nanoparticles).

The carbon has high specific surface area and good disperse-ability in the solvent and preferably fractal shape structures.

Several factors can affect the porosity or density of the reticulated film composites such as reducing solids in the slurry (i.e. from 10% to 6%) yields a few percent higher porosity, a higher drying temperature (i.e. 180° C. instead of 100° C.) increases porosity by few percent, a higher MW resin produces a higher porosity, a higher surface area filler makes a higher porosity. All these tunable properties can be applied to produce a reticulated film composite with a desired properties for a specific application.

### Application

The reticulated film composite of this invention can regulate hot spots inside the devices by slowing down electrical current.

The reticulated film composite of this invention is highly flexible and deformable electrically conductive films for wearable electronics or biomedical sensors.

The reticulated film composite of this invention can be used as a diffusion layers in fuel cells.

The reticulated film composite of this invention can be used as host in anode or cathode of lithium ion batteries or super capacitors.

The reticulated film composite of this invention can be used an electromagnetic interference, EMI, or radiofrequency interference, RFI, shielding.

The reticulated film composite of this invention can be used as catalyst support

The reticulated film composite not only does not shrink at elevated temperatures but also can be tuned to expand at hot spots inside the devices in order to slow down electrical current.

Another advantage of reticulated film composite is that can be cast on different surfaces and act as conductive network. Highly flexible and deformable electrically conductive films are useful for wearable electronics or biomedical sensors.

Another example, a reticulated film composite of PVDF and conductive carbons with 50% porosity can have applications in the energy storage, for instance, as bipolar plate coating or as diffusion layers in fuel cells, as host in anode or cathode of lithium ion batteries, and can provide long cycle life i.e. in lithium-sulfur batteries. The composite of this invention also can be used as high efficiency electrode in supper capacitors since possess an extremely large surface area.

Another example, a reticulated film composite of PVDF and conductive carbons with 50% porosity can have applications as a gas diffusion layers that are key components in various types of fuel cells, including Proton Exchange Membrane (PEM), and Direct Methanol (DMFC) and Phosphoric Acid (PAFC) stacks. The gas diffusion layers are placed on either side of the membrane in a fuel cell to allow uniform flow of reactants such as H2, air/oxygen, methanol, and product gases to pass evenly through it.

The reticulated film composite of this invention can have other applications such as an effective light weight electromagnetic interference, EMI, or radiofrequency interference, RFI, shieldings or shielding gaskets for electronic equipment, especially those used in aeronautics, especially because PVDF is UV and radiation resistance.

A reticulated film composite can also be used as catalyst support to provide high surface media for catalytically driven reactions and improve catalyst efficiency. The catalyst can be incorporated into reticulated film or can be deposited on it.

### Application

The response to temperature can be tuned with resin composition, For example varying the amount of HFP comonomer in PVDF resin because a reticulated film composite made of a higher HFP (i.e. 20% HFP) content resin will swell/expand at lower temperature relative to those with lower HFP (i.e. 8% HFP) content which may require a higher temperature to obtain the same swelling/expansion. Preferred weight percent of HFP in a copolymer of VDF is from 1 to 25 wt %, higher percentages of HFP can be used as high as 50 wt. %).

The coating can be cast on a substrate, it may be removed from the substrate and placed on another substrate or alternatively can be cast in conjunction with another layer in a wet on wet process.

One advantage of reticulated film composite is that can be simultaneously cast with another layer, i.e. using a double slot die casting machine to cast two slurry layers at the same time using a wet-on-wet technique. An integrated structure can be subsequently formed during the drying and calendaring steps. For multilayer composite structures, like electrode separators in an electrochemical device or filter media, can be cast wet on wet. When using the wet-on-wet technique the two layers become intertwined with no abrupt interfaces resulting in better adhesion. The reticulated film or coating can be cast simultaneously with and directly onto a substrate in a one step wet on wet process.

### Coating

In one embodiment, the carbon based nano particles or fibers may be surface treated, chemically (such as by etching or functionalization), mechanically, or by irradiation (such as by plasma treatment).

The particles are nano size. Preferably fibers have diameters below 1 micron.

The carbon based nano particles are present in the coating composition at 20 to 95 weight percent, and preferably 20-90 weight percent, based on the total of polymer solids and carbon based nano particles. When the content of the carbon based nano particle is less than 20 weight percent, the binder polymer is present in such a large amount as to decrease the interstitial volume formed among the particles.

Another example, a reticulated film composite can be used as protector coating, i.e. it exhibits high UV blocking/protection when nano size ZnO or nano-TiO₂ is included.

A reticulated film composite can also be used as catalyst support to provide high surface media for catalytically driven reactions and improve catalyst efficiency. The catalyst can be incorporated into reticulated film or can be deposited on it.

### Coating Method

The coating composition is applied onto at least one surface of a substrate by means known in the art, such as by brush, roller, ink jet, dip, knife, gravure, wire rod, squeegee, foam applicator, curtain coating, vacuum coating, slot die or spraying. The coating is then dried onto the Substrate at room temperature, or at an elevated temperature. The final dry coating thickness is from 0.5 to 500 microns, preferably from 1 to 100 microns, and more preferably from 2 to 50 microns in thickness.

In some aspects the reticulated film composite can be simultaneously cast with another layer.

### Aspects of the Invention

Aspect 1: A reticulated coating or film comprising a) a resin and b) nanoparticles, wherein the coating or film has a porous structure wherein the porous structure is from 10% to 80% open pores, wherein the resin has a solution viscosity of from about 100 cp to 10,000 cp, preferably from 100 cp to 5000 cp (measured at 5 wt % in NMP or at 2% water for water solution polymers, at room temperature) wherein the nanoparticles are carbon based and have a surface area of between 1 to 10000 m²/g, preferably 1 to 5000 m²/g, preferably 1 to 1000 m²/g, and wherein the film exhibits a recovery of thickness or porosity after being compressed and then heated of at least 30%, preferably 50%, preferably 55%, preferably 60%, preferably 70%.

Aspect 2: The reticulated coating or film of aspect 1 wherein the average pore size is less than 500 nm, preferably less than 100 nm, and more preferably less than 50 nm.

Aspect 3: The reticulated coating or film of aspect 1 or aspect 2 wherein the resin is selected from the group consisting of polyvinylidene fluoride (PVDF), PVDF-copolymers, poly ethylene-tetrafluoride ethylene (PETFE), polyvinyl fluoride (PVF), poly acrylates, poly methacrylates, poly styrene, poly vinyl alcohol (PVOH), polyesters, polyamides, poly acrylonitrile, poly acrylamide, carboxymethyl cellulose CMC, polyacrylic acids (PAA), polymethacrylic acids (PMAA), and their copolymers and combinations thereof.

Aspect 4: The reticulated coating or film of any one of aspects 1 to 3 wherein the resin comprises polyvinylidene fluoride homopolymer or copolymer.

Aspect 5: The reticulated coating or film of any one of aspects 1 to 3 wherein the resin comprises poly methacrylates.

Aspect 6: The reticulated coating or film of any one of aspects 1 to 3 wherein the resin comprises carboxymethyl cellulose.

Aspect 7: The reticulated coating or film of any one of aspects 1 to 3 wherein the resin comprises polyacrylic acid and/or polymethacrylic acid.

Aspect 8: The reticulated coating or film of any one of aspects 1 to 7 wherein the nanoparticles is selected from the group consisting of graphene, carbon nanotubes, conductive carbon, activated carbon and mixtures thereof.

Aspect 9: The reticulated coating or film of any one of aspects 1 to 7 wherein the nanoparticles comprise conductive carbon.

Aspect 10: The reticulated coating or film of any one of aspects 1 to 7 wherein the nanoparticles comprise activated carbon.

Aspect 11: The reticulated coating or film of any one of aspects 1 to 10 wherein the weight percent of polymer to nanoparticles is from 80:20 to 10:90, preferably 70:30 to 20:80.

Aspect 12: The reticulated coating or film of any one of aspects 1 to 11 wherein the nanoparticles have a surface area of from 1 to 700 m²/g, more preferably 1 to 600 m2/g.

Aspect 13: The reticulated coating or film of any one of aspects 1 to 12 wherein the coating has a thickness of from 0.1 to 500 microns, preferably from 0.5 to 100 microns, and more preferably from 0.5 to 50 microns, and more preferably from 0.5 to 20 microns.

Aspect 14: The reticulated coating or film of any one of aspects 1 to 13 wherein the nanoparticle size is less than 500 nm preferably less than 200 nanometers.

Aspect 15: The reticulated coating or film of any one of aspects 1 to 13 wherein the nanoparticle size is less than 100 nm.

Aspect 16: A method of making a reticulated coating or film, the method comprising the steps of
providing a resin dissolved in a solvent wherein the polymer has molecular weight as measured by solution viscosity of from about 100 cp to 10000 cp, preferably from 100 cp to 5000 cp (at 5 wt % in NMP or at 2 wt % in water for water soluble polymers, at room temperature),
providing nanoparticles, wherein the nanoparticles have surface area of 1 to 10000 m²/g,
combining the resin solution and the nanoparticles to produce a slurry wherein the weight percent of polymer to the weight percent of nanoparticle is from 80:20 to 5:95,
casting the slurry to form a coating or film,
drying the formed coating or film
   wherein the coating or film after drying has a porous structure wherein the porous structure is from 10% to 80% open pores, wherein the slurry exhibits a yield stress of between 50 dyne/cm2 and 5000 dyne/cm2, preferably between 75 to 3000 dyne/cm2, and wherein the solids content of the slurry is from 2 to 30 weight percent solids, preferably between 2 and 20 weight percent solids and wherein the film exhibits a recovery of thickness or porosity after being compressed and then heated of at least 30%, preferably 50%, preferably 55%, preferably 60%, preferably 70%.

Aspect 17: The method of aspect 16 wherein the average pore size is less than 1000 nanometers

Aspect 18: The method of aspect 16 wherein the average pore size is less than 100 nanometers, and more preferably less than 10 nanometers.

Aspect 19: The method of any one of aspects 16 to 18 wherein the resin is selected from the group consisting of polyvinylidene fluoride (PVDF), PVDF-copolymers, poly ethylene-tetrafluoride ethylene (PETFE), polyvinyl fluoride (PVF), poly acrylates, poly methacrylates, poly styrene, poly vinyl alcohol (PVOH), polyesters, polyamides, poly acrylonitrile, poly acrylamide, carboxymethyl cellulose CMC, polyacrylic acids (PAA), polymethacrylic acids (PMAA), and their copolymers and combinations thereof.

Aspect 20: The method of any one of aspects 16 to 18 wherein the resin comprises polyvinylidene fluoride homopolymer or copolymer.

Aspect 21: The method of any one of aspects 16 to 18 wherein the resin comprises poly methacrylates.

Aspect 22: The method of any one of aspects 16 to 18 wherein the resin comprises carboxymethyl cellulose.

Aspect 23: The method of any one of aspects 16 to 18 wherein the resin comprises polyacrylic acid and/or polymethacrylic acid.

Aspect 24: The method of any one of aspects 16 to 23 wherein the nanoparticles are selected from the group consisting of graphene, carbon nanotubes, conductive carbon, activated carbon or mixtures thereof.

Aspect 25: The method of any one of aspects 16 to 23 wherein the nanoparticles comprise conductive carbon or activated carbon.

Aspect 26: The method of any one of aspects 16 to 23 wherein the nanoparticles comprise graphene, or carbon nanotubes.

Aspect 27: The method of any one of aspects 16 to 26 wherein the solvent is selected from the group consisting of water, N-methyl-2-pyrrolidone (NMP), toluene, tetrahydrofuran (THF), acetone and hydrocarbons.

Aspect 28: The method of any one of aspects 16 to 26 wherein the solvent is selected from the group consisting of NMP, water, acetone and combination thereof, preferably NMP.

Aspect 29: The method of any one of aspects 16 to 26 wherein the solvent comprises water.

Aspect 30: The method of any one of aspects 16 to 26 wherein the solvent comprises NMP.

Aspect 31: The method of any one of aspects 16 to 30 wherein the solids content of the slurry formed containing both the solvent and the nanoparticles is from 2 to 15 weight %.

Aspect 32: The method of any one of aspects 16 to 30 wherein the solids content of the slurry formed containing both the solvent and the nanoparticles is from 2 to 12 weight percent.

Aspect 33: The method of any one of aspects 16 to 32 wherein the weight percent of polymer to the weight percent of nanoparticle is from 80:20 to 10:90.

Aspect 34: The method of any one of aspects 16 to 32 wherein the weight percent of polymer to the weight percent of nanoparticle is from 70:30 to 20:80,

Aspect 35: The method of any one of aspects 16 to 34 wherein the nanoparticles have a surface area of from 1 to 700 m²/g, more preferably 1 to 600 m2/g.

Aspect 36: The method of any one of aspects 16 to 34 wherein the coating has a thickness of from 0.1 to 100 microns, preferably from 0.5 to 50 microns, and more preferably from 0.5 to 20 microns.

Aspect 37: The method of any one of aspects 16 to 36 wherein the nanoparticle size is less than 500 nm preferably less than 200 nanometers.

. Aspect 38: The method of any one of aspects 16 to 36 wherein the nanoparticle size is less than 100 nm.

Aspect 39: The method of any one of aspects 16 to 36 wherein the film exhibits a recovery of thickness or porosity after being compressed and then heated of at least 55%, preferably at least 60%.

Aspect 40: The method of any one of aspects 16 to 39 wherein the reticulated film or coating is simultaneously cast directly with the substrate in one step in a wet on wet process.

Aspect 41: The reticulated coating or film made by the method of any one of aspects 16 to 40.

Aspect 42. An article comprising the reticulated coating or film of any one of aspects 1 to 15 and 41 wherein the article is selected from the group consisting of a separator in a wearable electronics or biomedical sensor, diffusion layer in a fuel cell, an electrochemical device such as anode or cathode of lithium ion batteries or super capacitors, electromagnetic interference, EMI, or radiofrequency interference, RFI, shielding and a catalyst support.

Aspect 42. An article comprising the reticulated coating or film of any one of aspects 1 to 15 and 41 wherein the article comprises an electrochemical device.

Aspect 43. An article comprising the reticulated coating or film of any one of aspects 1 to 15 and 41 wherein the article comprises a diffusion layer in a fuel cell.

Aspect 44. An article comprising the reticulated coating or film of any one of aspects 1 to 15 and 41 wherein the article comprises a diffusion layer in a catalyst support.

### Test Methods

Melt viscosity measured according to ASTM method D-3835 measured at 232° C. and 100 sec−1.

Particle size of nano particles can be measured using a Malvern Masturizer 2000 particle size analyzer. The data is reported as weight-average particle size (diameter).

Powder/latex average discrete particle size can be measured using a NICOMP™ 380 submicron particle sizer using laser light scattering. The data is reported as weight-average particle size (diameter).

Density of composite was calculated by dividing the weight of composite over volume of a specific sample. First the composite was cast on an aluminum foil, then a sample having 1.33 cm\hat{ }2 surface area was made by stamp cutting of the cast composite. The thickness of sample was measured with micrometer having accuracy of 0.1 micron. The weight of composite was measured using an analytical balance and subtracted the weight of the aluminum foil. Density of solid material is based on published literature values: i.e. PVDF polymers=1.78 g/cm3, PMMA=1.13 g/cm3, CMC=1.6 g/cm3.

BET specific surface area, pore volume, and pore size distribution of materials can be determined using a QUANTACHROME NOVA-E gas sorption instrument. Nitrogen adsorption and desorption isotherms are generated at 77K. The multi-point Brunauer-Emmett-Teller (BET) nitrogen adsorption method is used to characterize the specific surface area. A Nonlocal Density Functional Theory (NLDFT, N2, 77k, slit pore model) is used to characterize the pore volume and pore size distribution.

Solution viscosity: ASTM 2857

Yield stress back calculation: Brookfield Viscometer DV-III Ultra, spindle CP52 calculation based on the Herschel-Bulkley model equation:

τ=τº+kDⁿ

τ=Shear stress (D/cm2) k=Consistency index (cP) n=flow index
τº=Yield stress (D/cm2) D=Shear rate (1/sec)
τ=Shear stress (D/cm2): force tending to cause deformation of a material by slippage along a plane or planes parallel to the imposed stress.
τº=Yield stress (D/cm2): Yield stress is the amount of stress that an object needs to be permanently deformed or start flowing.
k=Consistency index (cP): related to the nature of the fluid. As the fluid becomes more viscous, consistency index increases.
D=Shear rate (1/sec): Shear rate is the rate of change of velocity at which one layer of fluid passes over an adjacent layer.
n=flow index: Flow behavior of complex fluids is traditionally characterized through the distinction between Newtonian and non-Newtonian fluids based on each their viscosity dependences on the rate of deformation and the change of shear rate.
τ is the shear stress, it needs to be divided by the shear rate to get the viscosity. The calculation would be:

$$\frac{{τ}^{°} + ( \frac{k}{100} ) {D}^{n}}{D} \times 100 = Viscosity ( cP )$$

k In the table is expressed in Centipoise so it needs to be divided by 100 to get it in D/cm2 and to add it to τº. To back calculate τº, the equation becomes

$${τ}^{°} = \frac{( Viscosity \times D )}{100} - ( \frac{k}{100} ) {D}^{n}$$

### Volume Resistivity Measurement:

Slurry is cast on aluminum foil with a thickness of about 110 micron and was placed in a convection oven for 30 min at 120° C. The Instron machine is then used with 3.09 cm2 gold plated electrodes to determine resistivity under different compression forces. The circular gold-coated contacts were adhered to fixture on Instron using 3M double-sided tape. Resistance was measured using Yokogawa digital resistance meter (755601, 4 probe). Contact pressure is applied using Instron (500N load cell) at rate of 20 N/min. All data was recorded manually. Resistivity decrease with pressure and around 100 N reaches a plateau.

$$Resistivity , ρ = \frac{R * A}{δ\_ eff} = ( Ω - c m )$$

where A=apparent contact area, cm2
R=measured resistance, Ω
δ_eff=effective thickness

### EXAMPLES

Example 1—Recovery from compression after calendaring for reticulated film composites of PVDF (Kynar 1810) copolymer of PVDF/HFP with 50 wt % HFP and PMMA (with RV=1.1 dl/g) resins using different conductive carbons and using fumed alumina as counter example.

| | Thickness (um) | | |
|---|---|---|---|
| | After drying | | After calendaring + |
| | (120° C. | After | 10 min at |
| | for 30 min) | calendaring | 150° C. |
| PVDF + Carbon | 10.5 | 5.5 | 7.5 |
| (SuperP) | | | |
| PVDF + Carbon | 11 | 6 | 8 |
| (Denka 100) | | | |
| PVDF + Carbon | 11 | 6 | 8 |
| (Denka 435) | | | |
| PVDF + Al2O3 | 12.5 | 4.5 | 4.5 |
| (counter example) | | | |
| PMMA + Carbon | 11.5 | 6.5 | 9.5 |
| (SuperP) | | | |
| PMMA + Al2O3 | 7 | 3.5 | 3.5 |
| (counter example) | | | |

This show that recovery is present for carbon based materials but didn't occur with Al₂O₃. A recovery of greater than 30% of original volume is observed upon heating.

Example 2 Effect of temperature on reticulated film composites:

| | | Masse of | Mass | | Density | Calculated | |
|---|---|---|---|---|---|---|---|
| | | 1.33 cm2 of | loading | Thickness | of Film | Solid | Porosity |
| | | coating | (mg/cm2) | (um) | (g/cm3) | density | (%) |
| 60% | Dried at 80° c. | 6.32 | 0.789 | 11 | 0.718 | 1.96 | 63.4 |
| Denka | Dried at 100° c. | 6.38 | 0.835 | 12 | 0.695 | 1.96 | 64.5 |
| 100 + | Dried at 120° c. | 6.22 | 0.714 | 11 | 0.649 | 1.96 | 66.9 |
| HSV-1810 | Dried at 180° c. | 6.43 | 0.872 | 13 | 0.671 | 1.96 | 65.8 |
| | Dried at 120° | 6.39 | 0.842 | 6 | 1.404 | 1.96 | 28.4 |
| | C. + Calendared | | | | | | |
| | Dried at 120° | 6.29 | 0.767 | 8 | 0.959 | 1.96 | 51.1 |
| | c. + calendared + | | | | | | |
| | 10 min 150° C. | | | | | | |
| | Dried at 80° c. | | | 7.5 | | 1.96 | |
| | Dried at 100° c. | 6.17 | 0.677 | 11.5 | 0.588 | 1.96 | 70.0 |
| | Dried at 120° c. | 6.11 | 0.632 | 11 | 0.574 | 1.96 | 70.7 |
| | Dried at 180° c. | 6.2 | 0.699 | 12.5 | 0.559 | 1.96 | 71.4 |
| | Dried at 120° | 6.3 | 0.774 | 6 | 1.291 | 1.96 | 34.1 |
| | C. + Calendared | | | | | | |
| | Dried at 120° | 6.31 | 0.782 | 8 | 0.977 | 1.96 | 50.1 |
| | c. + calendared + | | | | | | |
| | 10 min 150° C. | | | | | | |

This shows that porosity of greater than 50% or the original porosity is regained upon heating.

Resistivity Measurements:

Slurry were comprise of NMP (from Aldrich), conductive carbon super-P (from Timcal), and 3 different PVDF resins including Kynar® HSV-900 (from Arkema), Solef-5130 (from Solvay), and Kynar® HSV-1810 (from Arkema). Three composites were casted onto aluminum foil followed by drying in convection oven at 120 C. The resultant composites exhibited following volume resistivity.

Average volume resistivity of Super-P and its standard deviation using different binders

| | Av volume |
|---|---|
| Polymer | resistivity (Ω · cm) |
| HSV900 | 699 |
| 5130 | 1015 |
| HSV1810 | 1126 |

It appears that reproducibility of resistivity measurements are relatively good and any differences beyond 100 (Ω·cm) should be considered significant.

1. A reticulated coating or film comprising a) a resin and b) nanoparticles, wherein the reticulated coating or film has a open porous structure wherein the porous structure is from 10 vol % to 80 vol % open pores, wherein the resin has a solution viscosity of from about 100 cp to 10,000 cp, preferably from 100 cp to 5000 cp (measured at 5 wt % in NMP or at 2 wt % for water solution polymers, at room temperature) wherein the nanoparticles are carbon based and have a surface area of between 1 to 10000 m²/g, preferably 1 to 5000 m²/g, preferably 1 to 1000 m²/g, and wherein the film exhibits a recovery of thickness or porosity after being compressed and then heated of at least 30%, preferably 50%, preferably 55%, preferably 60%, preferably 70%.

2. The reticulated coating or film of claim 1 wherein the resin is selected from the group consisting of polyvinylidene fluoride (PVDF), PVDF-copolymers, poly ethylene-tetrafluoride ethylene (PETFE), polyvinyl fluoride (PVF), poly acrylates, poly methacrylates, poly styrene, poly vinyl alcohol (PVOH), polyesters, polyamides, poly acrylonitrile, poly acrylamide, carboxymethyl cellulose CMC, polyacrylic acids (PAA), polymethacrylic acids (PMAA) and their copolymers and combinations thereof.

3. The reticulated coating or film of claim 1 or 2 wherein the average pore size is less than 500 nm, preferably less than 100 nm, and more preferably less than 50 nm.

4. The reticulated coating or film of claim 1 wherein the resin comprises polyvinylidene fluoride homopolymer or copolymer.

5. The reticulated coating or film of claim 1 wherein the resin comprises poly methacrylates.

6. The reticulated coating or film of claim 1 wherein the resin comprises carboxymethyl cellulose.

7. The reticulated coating or film of claim 1 wherein the resin comprises polyacrylic acid and/or polymethacrylic acid.

8. The reticulated coating or film of any one of claims 1 to 2 wherein the nanoparticles is selected from the group consisting of graphene, carbon nanotubes, conductive carbon, activated carbon and mixtures thereof.

9. The reticulated coating or film of any one of claims 1 to 2 wherein the nanoparticles comprise conductive carbon.

10. The reticulated coating or film of any one of claims 1 to 2 wherein the nanoparticles comprise activated carbon.

11. The reticulated coating or film of any one of claims 1 to 2 wherein the weight percent of polymer to nanoparticles is from 80:20 to 10:90, preferably 70:30 to 20:80.

12. The reticulated coating or film of any one of claims 1 to 2 wherein the nanoparticles have a surface area of from 1 to 700 m²/g, more preferably 1 to 600 m2/g.

13. The reticulated coating or film of any one of claims 1 to 2 wherein the coating has a thickness of from 0.1 to 500 microns, preferably from 0.5 to 100 microns, and more preferably from 0.5 to 50 microns, and more preferably from 0.5 to 20 microns.

14. The reticulated coating or film of claim 12 wherein the nanoparticle size is less than 500 nm, preferably less than 200 nanometers.

15. The reticulated coating or film of claim 12 wherein the nanoparticle size is less than 100 nm.

16. A method of making a reticulated coating or film, the method comprising the steps of
a) providing a resin dissolved in a solvent wherein the polymer has a solution viscosity of from about 100 cp to 10000 cp, preferably from 100 cp to 5000 cp (at 5 wt % in NMP or at 2 wt % in water for water soluble polymers, at room temperature),
b) providing nanoparticles, wherein the nanoparticles have surface area of 1 to 10000 m²/g,
c) combining the resin solution and the nanoparticles to produce a slurry wherein the weight percent of polymer to the weight percent of nanoparticle is from 80:20 to 5:95,
d) casting the slurry to form a coating or film on a substrate,
e) drying the formed coating or film
wherein the coating or film after drying has a porous structure wherein the porous structure is from 10 vol % to 80 vol % open pores, wherein the slurry exhibits a yield stress of between 50 dyne/cm2 and 5000 dyne/cm2, preferably between 75 to 3000 dyne/cm2, and wherein the solids content of the slurry is from 2 to 30 weight percent solids, preferably between 2 and 20 weight percent solids and wherein the film exhibits a recovery of thickness or porosity after being compressed and then heated of at least 30%, preferably 50%, preferably 55%, preferably 60%, preferably 70%.

17. The method of claim 16 wherein the average pore size is less than 1000 nanometers

18. The method of claim 16 wherein the average pore size is less than 100 nanometers, and more preferably less than 10 nanometers.

19. The method of any one of claims 16 to 17 wherein the resin is selected from the group consisting of polyvinylidene fluoride (PVDF), PVDF-copolymers, poly ethylene-tetrafluoride ethylene (PETFE), polyvinyl fluoride (PVF), poly acrylates, poly methacrylates, poly styrene, poly vinyl alcohol (PVOH), polyesters, polyamides, poly acrylonitrile, poly acrylamide, carboxymethyl cellulose CMC, polyacrylic acids (PAA), polymethacrylic acids (PMAA) and their copolymers and combinations thereof.

20. The method of any one of claims 16 to 17 wherein the resin comprises polyvinylidene fluoride homopolymer or copolymer.

21. The method of any one of claims 16 to 17 wherein the resin comprises poly methacrylates.

22. The method of any one of claims 16 to 17 wherein the resin comprises carboxymethyl cellulose.

23. The method of any one of claims 16 to 17 wherein the resin comprises polyacrylic acid and/or polymethacrylic acids.

24. The method of claim 19 wherein the nanoparticles are selected from the group consisting of graphene, carbon nanotubes, conductive carbon, activated carbon or mixtures thereof.

25. The method of any one of claims 16 to 17 wherein the nanoparticles comprise conductive carbon or activated carbon.

26. The method of any one of claims 16 to 17 wherein the nanoparticles comprise graphene, or carbon nanotubes.

27. The method of claim 26 wherein the solvent is selected from the group consisting of water, N-methyl-2-pyrrolidone (NMP), toluene, tetrahydrofuran (THF), acetone and hydrocarbons.

28. The method of claim 24 wherein the solvent is selected from the group consisting of NMP, water, acetone and combination thereof, preferably NMP.

29. The method of claim 24 wherein the solvent comprises water.

30. The method of claim 24 wherein the solvent comprises NMP.

31. The method of claim 24 wherein the solids content of the slurry formed containing both the solvent and the nanoparticles is from 2 to 30%, preferably from 2 to 15 weight %.

32. The method of claim 24 wherein the solids content of the slurry formed containing both the solvent and the nanoparticles is from 2 to 12 weight percent.

33. The method of any one of claims 16 to 17 wherein the weight percent of polymer to the weight percent of nanoparticle is from 80:20 to 10:90.

34. The method of any one of claims 16 to 17 wherein the weight percent of polymer to the weight percent of nanoparticle is from 70:30 to 20:80.

35. The method of any one of claims 16 to 17 wherein the nanoparticles have a surface area of from 1 to 700 m²/g, more preferably 1 to 600 m2/g.

36. The method of any one of claims 16 to 17 wherein the coating has a thickness of from 0.1 to 100 microns, preferably from 0.5 to 50 microns, and more preferably from 0.5 to 20 microns.

37. The method of claim 24 wherein the nanoparticle size is less than 500 nm preferably less than 200 nanometers.

38. The method of any one of claims 16 to 17 wherein the nanoparticle size is less than 100 nm.

39. The method of any one of claims 16 to 17 wherein the film exhibits a recovery of thickness or porosity after being compressed and then heated of at least 55%, preferably at least 60%.

40. The method of any one of claims 16 to 17 wherein the reticulated film or coating is simultaneously cast directly with the substrate in one step in a wet on wet process.

41. The reticulated coating or film made by the method of any one of claims 16 to 40.

42. An article comprising the reticulated coating or film of any one of claims 1 to 15 and 41 wherein the article is selected from the group consisting of a separator in a wearable electronics or biomedical sensor, diffusion layer in a fuel cell, an electrochemical device such as anode or cathode of lithium ion batteries or super capacitors, electromagnetic interference, EMI, or radiofrequency interference, RFI, shielding and a catalyst support.

43. An article comprising the reticulated coating or film of any one of claims 1 to 15 and 41 wherein the article comprises an electrochemical device.
